(19) United States
(12) Patent Application Publication
(10) Pub. No.: US 2023/0249005 A1
(43) Pub. Date: Aug. 10, 2023

(54) Low-Intensity Pulsed Ultrasound Enhancement of Testosterone Production

(71) Applicant: The Regents of the University of California, Oakland, CA (US)

(72) Inventors: Tom Futai Lue, Hillsborough, CA (US); Guiting Lin, San Francisco, CA (US)

(21) Appl. No.: 18/015,688

(22) PCT Filed: Jul. 28, 2021
(86) PCT No.: PCT/US2021/043549

§ 371 Date: Jan. 11, 2023

Related U.S. Application Data
(60) Provisional application No. 63/057,516, filed on Jul. 28, 2020.

(51) Int. Cl. A61N 7/00

(52) U.S. Cl. CPC A61N 7/00

(57) ABSTRACT

Provided are novel dosages for the safe and efficacious application of low intensity pulsed ultrasound (LIPUS) energy to increase testosterone in a subject. These newly discovered dosage ranges specify therapeutically effective dosages that avoid harmful and inconsistent effects of LIPUS as observed in prior art attempts to implement this technology platform. Disclosed is a novel calculation of a single, biologically relevant measure of LIPUS energy dosage. By the novel integrated dosage calculations described herein, the art is provided with a tool for comparing studies and standardizing doses across a range of instruments and administration protocols. Also provided are novel medical devices and ultrasonic applicators that enable the facile administration of LIPUS to testes of subjects.

### CROSS-REFERENCE TO RELATED APPLICATIONS

This application claims the benefit of priority to U.S. Provisional Patent Application Ser. No. 63/057,516 entitled “Low-intensity Pulsed Ultrasound Enhancement of Testosterone Production,” filed Jul. 28, 2020, the contents of which are hereby incorporated by reference.

### STATEMENT REGARDING FEDERALLY SPONSORED RESEARCH OR DEVELOPMENT

Not Applicable

### BACKGROUND OF THE INVENTION

The steroid molecule testosterone is the primary sex hormone in males. Testosterone is active in a large array of physiological processes, including sexual development and fertility, sexual function, maintenance of muscle mass and bone density, and others. In various contexts, reduced production of testosterone below normal physiological levels can occur. In some cases, injury or disease affecting the testes, pituitary gland, or brain can cause low testosterone. Additionally, a natural and substantial reduction in testosterone production occurs with increasing age after middle age.

Low testosterone has been treated by the application of exogenous androgens, referred to as testosterone therapy. Many forms of exogenous androgens have been developed, including bioidentical and synthetic androgens. Depending on the formulation, application may be transdermal, oral, by injection or implantation through a small incision on the skin.

Testosterone therapy has been investigated, and in some cases is approved for, treating various conditions associated with male hypogonadism such as erectile dysfunction, muscle wasting, and improving bone density. Additionally, there is high interest in the application of testosterone therapy for increasing testosterone levels in aged subjects, for both health and quality of life effects. However, various studies have linked administration of testosterone to increased risk of adverse cardiovascular events such as stroke or heart attacks. The United States Food and Drug Administration has issued an advisory that prescription testosterone products are approved only for men who have low testosterone levels caused by specified medical conditions, and that the benefit and safety of these agents has not been established for the treatment of low testosterone levels due to aging.

Accordingly, with the safety of exogenous hormone application being unclear, there is a high demand for novel methods of boosting endogenous testosterone production. Additionally, there is a need in the art for methods of increasing testosterone conveniently and without prescription.

One potential means of raising endogenous testosterone levels that has been explored is the application of ultrasound to the testes in order to stimulate testosterone production. Haddid et al. (Effect of low-intensity pulsed ultrasound on prepubertal rat testis, Braz J Med Biol Res. 1991; 24(7):697-700) describe the application of low-intensity pulsed ultrasound (LIPUS) to juvenile rat testes with a resulting increase in plasma testosterone, an increase in seminal vesicle relative weight, and an increase in the fructose and DNA contents of the gland. However, in a follow-up study [Haddid et al., Low-intensity ultrasound energy applied to the testes of aged rats, 1998, Histol Histolpathol 13: 385-389] when LIPUS was applied to the testes of aged rats, a substantial reduction in testosterone levels and sperm production was observed.

In light of these contradictory results, the utility of LIPUS for increasing endogenous testosterone production is unclear and there is a need in the art for determining the actual utility of the therapy, especially in aged males. In light of the detrimental effects observed in the previous study of aged rats, there is also a need in the art for novel LIPUS protocols that are safe and effective in mature and aged subjects.

### SUMMARY OF THE INVENTION

The inventors of the present disclosure have determined that LIPUS may be safely applied in male subjects, including aged males, to substantially increase testosterone production. Furthermore, the inventors of the present disclosure have determined the range of physiologically effective LIPUS dosages that may be applied without adverse side effects. The novel inventions disclosed herein provide the art with a local, non-invasive and conveniently applied therapy to elevate natural testosterone level in the body to normal physiologic ranges while avoiding the adverse side effects associated with current testosterone replacement therapies.

In one aspect, the scope of the invention encompasses methods for the safe and efficacious application of LIPUS to increase testosterone production in male. In one aspect, the scope of the invention encompasses methods for increasing and activating Leydig cells, to achieve an increase in testosterone production.

In one aspect, the scope of the invention encompasses methods for the safe and efficacious application of LIPUS in hypogonadal subjects. In one embodiment, the hypogonadal subject is an aged subject, for example, treated to attenuate or compensate for age-related decreases in testosterone production. In one embodiment, the subject is a hypogonadal male in need of treatment for obesity-relate hypogonadism.

In one aspect, the scope of the invention encompasses novel dosages for the safe and efficacious application of LIPUS to increase testosterone in subject. These newly discovered dosage ranges specify therapeutically effective dosages that avoid harmful and inconsistent effects of LIPUS as observed in prior art attempts to implement this technology platform.

In one aspect, the scope of the invention encompasses the calculation of a single, biologically relevant measure of LIPUS energy dosage. By the novel integrated dosage calculations described herein, the art is provided with a tool for comparing studies and standardizing doses across a range of instruments and administration protocols

In one aspect, the scope of the invention encompasses novel treatment regimen. The inventors of the present disclosure have determined that the effects of applied LIPUS are not permanent. The have further determined that LIPUS may be administered at an optimized schedule of regular treatment intervals discovered to maintain testosterone with minimal treatment.

In one aspect, the scope of the invention encompasses novel medical devices and ultrasonic applicators that enable the facile administration of LIPUS to subjects.

In one aspect, the scope of the invention encompasses novel method of treating cells to activate therapeutically biological signaling pathways, including PERK/ATF4 and Wnt-β-catenin signaling pathways.

The various implementations of the invention are described next.

### BRIEF DESCRIPTION OF THE DRAWINGS

FIG. 1 depicts blood testosterone level (T) in young (3 months of age) and older adult rats (six months of age), treated with LIPUS at a dosage of 67 mW/cm² BE A first LIPUS treatment was administered over the course of 4 weeks, with eight applications, delivered every twice a week. T was monitored at 1 week, 6 weeks and 18 weeks after the completion of the initial LIPUS treatment. At 18 weeks, a second LIPUS treatment (six applications, administered twice a week for three weeks) was administered and T was measured again 1 week following completion of the second treatment

FIGS. 2A and 2B depict serum testosterone (FIG. 2A) and luteinizing hormone (LH) (FIG. 2B) levels before and following completion of LIPUS therapy in 3-month-old (3M, 3 months of age, n=6) and 6-month-old (6M, six months of age, n=6) male SD rats. LIPUS was administered at 67 mW/cm², 2 mins, twice a week for 4 weeks. Serum testosterone and LH was measured before (Pre) and 1 week (1 wk) after the last LIPUS application.

FIG. 3A-3D summarizes the results of a stereological study of Leydig cells following LIPUS therapy. The LIPUS treatment was applied to adult rats (6-month-old) at a dosage of 67 mW/cm², 2 mins, twice a week for 3 weeks. FIG. 3 is a bar chart of Leydig cell nuclear volume (LCNV); FIG. 3B depicts Leydig cell cytoplasmic volume (LCCV); FIG. 3C depicts Leydig cell total volume (LCTV); and FIG. 3D depicts Leydig cell absolute number (LCAN).

FIGS. 4A and 4B depict testicular blood vessels in older rats treated with LIPUS therapy. The LIPUS therapy was applied to older rats (10-month-old) at a dosage of 67 mW/cm², 2 mins, twice a week for 4 weeks. FIG. 4A shows blood vessel number per low-power field (LPF). FIG. 4B shows that Leydig cell absolute number (LCAN) per high-power field (HPF).

FIGS. 5A, 5B, and 5C. demonstrate that LIPUS activated Wnt and ISR signaling pathway in TM3 Leydig cells in vitro. The TM3 cells were treated with LIPUS at different energy levels at 67, 86, and 133 mW/cm² for 2 mins. 24 hours later the protein was isolated for western blotting. FIG. 5A: phosphorylation level of p-PERK was significantly increased after LIPUS at different energy levels (n=3,*P<0.05). FIG. 5B: IDV (integrated density value) ratio of ATF4 to β-actin was significantly increased after LIPUS at all 3 energy levels (n=3,*P<0.05). FIG. 5C: IDV ratio of β-catenin to β-actin was significantly increased after LIPUS at 67 mW/cm² level only (n=3, *P<0.05).

FIGS. 6A and 6B summarize the effects of LIPUS on serum testosterone and luteinizing hormone (LH) in older male rats. FIG. 6A depicts serum testosterone (a) and FIG. 6B depicts LH levels in control (ctrl, n=6) and LIPUS therapy (LIPUS, n=7) treatments were measured. (p<0.05).

FIG. 7A and FIG. 7B depict the age effect on Serum Testosterone levels following LIPUS therapy. Serum testosterone in 14M rats (FIG. 7A) and in 16M rats (FIG. 7B) levels 1 week (1 wk) after the last LIPUS therapy were measured.

FIG. 8 depicts cellular apoptosis induced by LIPUS at different LIPUS dosages, measured by TUNEL staining to identify apoptotic cells. Brz 1.5=20 mW/cm², 20 mins every day for 7 days; 1.7 MHz LIPUS (Brz 1.7)=20 mW/cm², 20 mins every day for 7 days; UCSF 1.5=, 20 mW/cm², 2 mins twice a week for 3 weeks; UCSF 1.7=20 mW/cm², 2 mins twice a week for 3 weeks.

FIG. 9 depicts serum testosterone concentrations following Brazilian and UCSF LIPUS therapy in 36 weeks old rats. Brz 1.5=20 mW/cm², 20 mins every day for 7 days; 1.7 MHz LIPUS (Brz 1.7)=20 mW/cm², 20 mins every day for 7 days; UCSF 1.5=, 20 mW/cm², 2 mins twice a week for 3 weeks; UCSF 1.7=20 mW/cm², 2 mins twice a week for 3 weeks.

FIG. 10 depicts the dosage response of serum testosterone concentrations in 12 weeks old rats following UCSF LIPUS therapy on testicles. LIPUS 1=67 mW/cm². LPUS 2=86 mW/cm².

FIG. 11 depicts Leydig cell counts in testicles treated with different dosages of LIPUS. Brz 1.5=20 mW/cm², 20 mins every day for 7 days; 1.7 MHz LIPUS (Brz 1.7)=20 mW/cm², 20 mins every day for 7 days; UCSF 1.5=, 20 mW/cm², 2 mins twice a week for 3 weeks; UCSF 1.7=20 mW/cm², 2 mins twice a week for 3 weeks.

FIG. 12 depicts administered dosages of biological energy (“BE”) vs. Power Density at varying power density instrument settings.

FIG. 13 depicts edema score vs. BE dosage value.

FIG. 14 depicts apoptotic index in response to different LIPUS BE dosages in rat testes.

FIG. 15 illustrates a method of administering a LIPUS treatment by means of a handheld instrument (104) comprising a LIPUS transducer (101) that can be readily positioned by hand. The treatment comprises two applications, one to each of the left (107) and right side (108) of the same testis

FIG. 16 depicts an exemplary configuration of the LIPUS device comprising a transducer (101) connected to power and control sources (not shown) by a wire (102). In this configuration, a water bladder (103) covers the transducer, wherein the water bladder receives and conforms to the shape of the scrotum (110).

FIG. 17 depicts an exemplary configuration of the LIPUS application device wherein the transducer (101) is covered by a water bladder (103), wherein the transducer and water bladder are configured as a cup or depression (105) configured to receive the scrotum and being integrated with a chair (106).

### DETAILED DESCRIPTION OF THE INVENTION

The methods of the invention provide the art with a novel means of boosting endogenous testosterone levels in a subject by the application of LIPUS to the testes. The general method of the invention encompasses a process as follows:
A method of treating a low testosterone condition in a subject in need of treatment therefor by the administration of a treatment comprising one or more LIPUS applications to the testes of the subject, wherein the one or more LIPUS applications is administered at a safe and efficacious dosage.

Each element of the general method will be described in detail next. In the following description, as used herein, “about” means within 5% plus or minus of the enumerated value.

Low Testosterone Conditions. The methods of the invention may be applied in the treatment of a low testosterone condition in a subject. The subject may be a male of any species. In a primary embodiment, the subject is a human male. In other embodiments, the subject may be a non-human animal such as a test animal, veterinary subject, livestock, or pet. Exemplary species include mice, rats, dogs, cats, cattle, horses, pigs, camels, or other species.

In one embodiment, the methods of the invention are applied in the treatment of a condition wherein the condition is low testosterone. Testosterone, as used herein refers to biologically available testosterone as measured by standard assays. In a primary embodiment, testosterone is measured as bioavailable testosterone in blood as measured by the Vermeulen calculation or modified Vermeulen method, as known in the art.

Low testosterone, as used herein means below-normal testosterone production in a subject, i.e. testosterone is below a selected threshold indicative of normal testosterone abundance for like subjects. For example, in one implementation, in human males, a blood testosterone level of 300 to 1,000 nanograms per deciliter (ng/dpl) may be considered normal. In some embodiments, low testosterone encompasses a testosterone blood level of less than 300 ng/dL. In some embodiments, the low testosterone threshold is selected based on the normal testosterone levels of like subjects, for example, subjects of the same age bracket or sharing other demographic characteristics. Low testosterone may be established according to a selected threshold value, wherein, if the subject's testosterone levels are below the threshold, the subject is considered to have low testosterone. The threshold may be established for a selected population or age bracket. In some implementations, a low testosterone level is a measured blood level of testosterone below 300 nanograms per deciliter. In other embodiments, low testosterone may be any of a measured blood concentration of testosterone below: 150 ng/dl; 200 ng/dl; 250 ng/dl; 350 ng/dl; 400 ng/dl; 450 ng/dl; 500 ng/dl or any other selected blood testosterone level.

In some embodiments, the condition is age-related low testosterone and the subject is an aged subject. In various embodiments, an aged human subject may be subject of at least 40 years, at least 45 years, at least 50 years, at least 55 years, or at least 60 years of age.

In some embodiments, the low testosterone condition is a condition caused by or characterized by lower than normal testosterone. Exemplary conditions include primary hypogonadism, including congenital or acquired hypogonadism. Such conditions include testicular failure due to cryptorchidism, bilateral torsion, orchitis, vanishing testis syndrome, orchiectomy, Klinefelter Syndrome, chemotherapy, or toxic damage from alcohol or heavy metals. Such subjects typically have low serum testosterone concentrations and gonadotropins (FSH, LH) above normal range. Exemplary conditions further include hypogonadotropic hypogonadism, including congenital or acquired hypogonadism, encompassing gonadotropin or luteinizing hormone-releasing hormone (LHRH) deficiency or pituitary-hypothalamic injury from tumors, trauma, or radiation. Such subjects typically have low testosterone serum concentrations but have gonadotropins in the normal or low range.

In other embodiments, the low testosterone condition is a condition wherein testosterone is below normal levels due to testicular injury or infection; chemotherapy or radiation treatment for cancer; corticosteroid or anabolic steroid use; hormone disorders such as pituitary tumors or abnormally high prolactin; or chronic diseases such as diabetes, kidney disease, or HIV infection; genetic disorders such as Klinefelter syndrome, Kallman syndrome, or hemochromatosis.

In another implementation, the low testosterone condition is low testosterone associated with obesity. In some implementations, the subject is an obese subject.

In various implementations, “treatment” encompasses the achievement of a selected therapeutic or biological effect. Exemplary biological or therapeutic effects include, for example: increasing testosterone levels in the subject, for example, increasing blood testosterone concentrations; restoring testosterone levels (e.g. blood testosterone concentration) to normal ranges; increasing blood testosterone levels to at least 300 ng/dl; increasing the proliferation rate of Leydig cells; increasing the size of Leydig cells; preventing or slowing declines in testosterone production; increasing the proliferation rate of Sertoli cells; improving sexual function; increasing muscle mass; improving sperm production; ameliorating or curing the symptoms of a selected condition; slowing, halting, or reversing the progression of a selected condition; or preventing the onset or reducing the probability of onset of a selected condition.

LIPUS. The LIPUS treatment encompasses the administration of ultrasound to the testes of the subject. Ultrasound encompasses mechanical vibrational energy beyond the upper limit of human hearing, for example, having a frequency of at least 15-20,000 Hz. The mechanisms by which ultrasound achieves therapeutic effects are not known, but may encompass transfer of energy to target tissues by sonically induced oscillations, which may induce beneficial gene expression, cell division, protein synthesis and other biological effects.

In a primary implementation, the methods of the invention encompass the administration of LIPUS ultrasound. LIPUS encompasses a low energy, pulsed waveform, which avoids substantial thermal effects induced by higher intensity continuous wave systems. For example, LIPUS may encompass the delivery of pulses with power density (PD) of less than 30 mW/cm².

LIPUS may be administered by any number of suitable instruments capable of delivering ultrasound dosages enumerated herein. Typical LIPUS systems will comprise a function generator, power amplifier and one or more ultrasonic transducers, for example, a piezoelectric transducer. Exemplary LIPUS instruments include the Exogen 2000 device (Smith & Nephew, Inc. London, UK); SAFHS device (Tajin Pharma, Tokyo, Japan); ST-SONIC (ITO Corporation Ltd., Tokyo, Japan); Duson device (SRA Developments, UK); and the A392S device (Parametric, Waltham, Mass., USA).

LIPUS application to the testes may be achieved by contacting the scrotum with the one or more transducers of the LIPUS system, for example, an array of transducers. In some implementations, specialized devices of the invention, described further herein, are utilized to apply the ultrasonic energy to the scrotum. Typical applications will utilize a gel or other medium, as known in the art, to effectively transfer energy from the transducer to the scrotum.

The application may conveniently be a self-application. For example, devices for LIPUS administration may be used by a subject at home. In other embodiments, the application is by trained medical personnel, for example, at sites where the necessary instruments are available.

LIPUS Dosages.

The scope of the invention encompasses methods of delivering LIPUS within specified energetic dosage ranges that are both efficacious and safe. By extensive experimentation, the inventors of the present disclosure have determined the minimal dosage that is required to be received by the target organ, the testes, to promote a therapeutic effect (e.g. increased testosterone) and a maximum dosage that may be received by the target organ, the testes, without harm to the subject, wherein dosages higher than the maximum dosage may cause damage to the testes of the subject. The discoveries disclosed herein were enabled by an extensive series of experiments, wherein the biological effects of a range of LIPUS dosages were monitored in cultured cells and in live animals, with application settings varied in a systematic fashion. It was not previously known how biological systems would respond as each of the LIPUS dosage variables were altered. The unprecedented survey of LIPUS dosage parameters on biological responses described herein allowed the inventors of the present disclosure to derive the relationship between each component of the energetic dose and the resulting physiological effects, and to define the bounds of a safe and efficacious LIPUS dosage.

In the practice of the invention, the subject will be administered a treatment of LIPUS. Each treatment comprises one or more applications of LIPUS to the testes of the subject. An “application,” as used herein, means an administration of LIPUS to the subject in a single session. The “application dosage” will refer to the total biological energy, as described below, that is received by the target organ, i.e. the testes, in the application. A treatment may comprise one or more applications, administered according to a selected treatment schedule, as described below.

The dosages disclosed herein are formulated for application to a single testis (also referred to as a “testicle”) of the testes. Accordingly, it will be understood that the disclosed application dosages are directed to just a single testis. The treatment methods disclosed herein contemplate the application of the selected dosages to each of the two testis. As described below, this may be achieved by two separate applications, one to each testis. In some implementations, a dosage is delivered to the entire scrotum, simultaneously treating both testes, in which case the recommended dosage per testis may be doubled or otherwise adjusted to account for attenuation.

The application dosage is a measure of the biologically relevant energy received by the testes (or other target organ as the case may be), and this will be equal to:

[Received dosage]=[the instrument dosage]×[an attenuation coefficient]. [Equation 1]

The instrument dosage means the energetic dosage administered (emitted) by the ultrasound transducer of the LIPUS instrument. In the case of a LIPUS instrument comprising multiple transducers, the instrument dosage means the dosage collectively administered (emitted) by multiple ultrasound transducers of the instrument. The attenuation coefficient is a measure of energetic attenuation of the administered energy by structures intervening between the ultrasonic transducer(s) of the LIPUS instrument and the target cells of the testes. Each of the instrument dosage and attenuation coefficients are described in detail next.

Calculating LIPUS Instrument Dosages. A first parameter in determining safe and efficacious dosage received by each testis is the instrument dosage. The instrument dosage may be defined as the biologically effective energy emitted by the LIPUS instrument. LIPUS instruments are configured to emit energy according to various parameters, including:
the power density of the ultrasonic pulses (PD), which is expressed in power per unit area, for example, milliwatts (mW) per cm²;
the frequency of pulse delivery (pulse frequency, PF), in Hertz, expressed in Hz or pulses per second;
the duration of the application (time, T), which can be expressed in seconds; and
the duty-cycle or pulse ratio (PR) of the pulse, which is the proportion of the time ultrasound signal is emitted (‘on”) over the course of the pulse duration, and which can be expressed as a percentage or decimal value between 0 and 1.

LIPUS instruments are typically configured such that each of the emission parameters may be independently adjusted, enabling delivery of a wide range of energetic dosages.

The scope of the invention encompasses novel LIPUS dosages that are therapeutically effective while avoiding supra-optimal energetic deliveries that cause damage or harm to the treated tissues. The inventors of the present disclosure have invented a convenient formulation that integrates LIPUS instrument parameters into a single, biologically relevant measure of instrument output. This is advantageous in that the multifactorial aspect of LIPUS administration has made it difficult to compare results from studies using different LIPUS settings, and therefore difficult to develop standard dosages. By the novel integrated dosage calculations described herein, the art is provided with a tool for comparing studies and standardizing doses across a range of instruments and administration protocols.

The integrated dosage described herein provides a measure of the physiologically relevant, cumulative LIPUS energy delivered in a LIPUS application, taking into account power density, pulse duty cycle, the duration of application, and the frequency of pulse application. The dosage is integrated, in that it reconciles all four components of LIPUS energy delivery.

The invention encompasses the calculation of LIPUS energy as biologically effective energy, or “Biological Energy” (BE), which represents the biologically relevant energy delivered in a LIPUS application. In one embodiment, BE is calculated as

BE=PD×T×PR×PF^{k} [Equation 2]

wherein: PD is power density per pulse in mW/cm²; T is the duration of the treatment in seconds; Duty cycle is the pulse ratio (PR) of the pulse, expressed as a value between 0 and 1; PF is frequency of pulse delivery, measured n Hz and expressed as a unitless value in the calculation of Equation 1; and k is the coefficient of pulse frequency (PF), described below. The units of BE may be expressed as watt seconds per square centimeter (WS/cm²).

BE is a frequency-adjusted, biologically effective measure of cumulative energy. The term (PF)^{k} acts like a constant in the equation, wherein the actual, physical energy delivered is PD×PR×T which yields total mW delivered per cm². The inventors of the present disclosure have discovered that the biological effects of the total energy delivered varies depending on how fast the energy is delivered, wherein the faster a given amount of energy is delivered, the stronger its biological effect. As formulated in Equation 1, energetic dosage is upwardly adjusted by a “speed factor”, being a fractional power function of the frequency: (PF)^{k}. For example, with regards to a BE reference value established at 1 MHz, when k is selected as 0.373, biologically effective energy delivered increases by about 30% at 2 MHz, by about 50% at 3 MHz and by about 80% at 5 MHz. The fact that PF is raised to a fractional power means that the units in which Hz is measured, pulses/sec, are also raised to that power and thereby lose meaning. Therefore, in the calculation of BE, the frequency term, determined in Hertz, should be expressed as a unitless value. For clarity, BE is a biologically effective frequency adjusted measure and is not a measure of cumulative energy as determined by physics. BE is an independent measure that has relevance only in the context of ultrasound treatments. It will be understood that the energy-biological dosage parameter relationship described in Equation 1 may be described by alternative mathematical formulas.

In some embodiments, k=0.373. In some embodiments, k may vary between 0.25 and 0.5, for example, in various embodiments being selected from 0.25, 0.26, 0.27, 0.28, 0.29, 0.30, 0.31. 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, and 0.50, or an intermediate value within the enumerated range. In some alternative implementations, k is 1 and the exponential is not used.

The integrated biologically effective dosage calculations of the invention, in one embodiment the BE value, provide the art with a novel method of formulating a therapeutically relevant LIPUS application dosage. Further, the methods of the invention allow, for the first time, a common dosage measure that accommodates definable ranges. Further, the methods of the invention allow, for the first time, a common dosage measure that accommodates any range of power densities, waveforms, treatment durations, and pulse application frequencies. This advantageously allows, for the first time, a convenient side-by-side comparison of dosages which vary in the four dosage parameters. This also allows the practitioner, for the first time, to optimize the four parameters of a LIPUS treatment session to achieve a desired dosage. For example, LIPUS applicator instruments typically are constrained by various settings, e.g. they can deliver ultrasound at a limited number of discreet power densities and can be set to a limited number of discreet pulse delivery rates. Using the dosage calculation methods of the present invention, the proper number of pulses to deliver using a LIPUS instrument's fixed power density and frequency settings can be determined.

Calculating Attenuation Coefficients. The energy emitted by the LIPUS instrument may be attenuated in its path to the target cells of the testes. Attenuation may be anatomical, clothing related, or due to instrument features disposed between the ultrasonic emitter(s) and the scrotum.

In one implementation, the instrument dosage and the energetic dosage received by the testes is treated as equal. In this implementation, the attenuation coefficient is 1.0 and attenuation effectively drops from the dosage calculation. In many implementations, this assumption is made because the testes are close to the surface of the scrotum and a substantial portion of cells in the testes may advantageously be accessed by ultrasonic energy applied to the scrotum without significant attenuation of the applied LIPUS energy.

Alternatively, in some embodiments, anatomical attenuation is factored into the dosage calculation. The treatment objective is to deliver a safe and efficacious dosage to the entire testes or internal cells within the oval-shaped structure of the testes. Due to the mass and diameter of these anatomical structures, in some cases attenuation of administered LIPUS energy may occur when delivering ultrasonic energy to the most internal cells of the testes. Accordingly, in some embodiments the LIPUS dosage emitted by the instrument is selected to account for attenuation of energy by the mass of the testes. Alternatively, in larger testicles, the application can be administered separately the from right and left side of the testicle with one half dosage applied to each, in order to compensate for the anatomical attenuation of ultrasound energy.

Ultrasound attenuation coefficients for a selected tissue are a function of the physical properties of the tissue, the waveform of the applied ultrasound, and the frequency. One of skill in the art may utilize attenuation coefficients of testicular tissue to calculate a dosage that is efficacious across the testes. In some implementations, for application of LIPUS to the testes, attenuation is estimated to be within the range of 30-50% per cm depth of tissue. In various embodiments, attenuation values of 30%, 31%, 32%, 33%, 34%, 35%, 36%, 37%, 38%, 39%, 40%, 41%, 42%, 43%, 44%, 45%, 46%, 47%, 48%, 49%, or 50% per cm are utilized, for example, for applications having a frequency in the range of 1-5 MHz, for example, 1-3 Mhz. In some embodiments, the selected attenuation value is 35-40% per cm, for example, 38% per cm.

In one implementation, attenuation in the range of 30-40% per cm is contemplated. As the testes are ovoid structures typically of more than one centimeter in diameter, effective treatment of the entire testis may assume attenuations of 30-60% in treatment of a single testis. Accordingly, instrument dosage may be adjusted upwards to compensate for the expected attenuation. In various implementations, instrument dosage is delivered at a value equal to 110%, 120%, 130%, 140%, 150%, 160%, 170%, 180%, 190%, 200%, 225%, 250%, 275%, 300% (or greater) of the desired dosage to be received by the testis. Care should be taken to assure that outer cells of the testis are not overtreated in applying an effective dosage to the inner cells of the testis.

In some implementations, the LIPUS instrument is applied to the bare skin of the scrotum, or with a minimal barrier between the instrument and scrotum (e.g. a layer of conductive gel and/or a sanitary disposable covering comprising paper or polymeric material. In other implementations, the application is applied through the clothing of the subject. In such case, the attenuating properties of clothing should be accounted for in determining the attenuation coefficient. In some implementations, clothing attenuates the applied ultrasonic energy by a factor of 0.95-0.75 (i.e. 5 to 25% of the applied energy is lost).

As described below, in some implementations, the LIPUS is applied to the testes by use of an ultrasonic emitter (or array of emitters) wherein a water bladder or other structure, such as pad, is interposed between the emitter(s) and the testes. In such implementations, an attenuation coefficient may be applied to account for the attenuation by these intervening structures of the instrument. This attenuation may be measured directly by methods known in the art for measurement of ultrasonic energy. Alternatively, the attenuation may be calculated based on the materials and physical configuration of the attenuating structures.

Safe and Effective LIPUS Dosage Applications for the Testes. The inventors of the present disclosure have determined a range of LIPUS application dosages which are both efficacious and safe. Notably, the inventors of the present disclosure have determined that the dosages applied in reported prior art studies were supra-optimal energetic dosages that exceeded the safe threshold for application of ultrasound to the testes.

In one embodiment, the efficacious and safe dosage is defined by a range of BE values.

An efficacious dosage means a dosage which imparts at least a minimal measurable therapeutic effect, for example, inducing any of: increasing Leydig cell numbers, size, or testosterone production.

A safe dosage is a dosage which avoid measurable harm or detrimental effects to the treated tissues. A safe dosage may be defined by various attributes, for example: a dosage that avoids causing significant edema in treated testes; a dosage that avoids causing significant apoptosis in cells of the testes, for example, Leydig cells, Sertoli cells, or other cells; a dosage that avoids causing a reduction in the proliferation rate of Leydig cells and Sertoli cells; a dosage that avoids causing hemorrhage; or a dosage that avoids causing any other selected measure of harm in the cells, tissues, and functions of the testes.

In one implementation, the scope of the invention encompasses administration to a testis of a LIPUS application wherein the cells (or a substantial proportion of the cells) of the testis receive an energetic dosage that is efficacious, for example, is above a selected minimum effective dosage threshold. In various implementations, the minimum effective dosage threshold may be a value selected in the range of 5-25 WS/cm² BE. In one implementation, the minimum efficacious LIPUS dosage may be a BE value of at least 10 WS/cm². At values below 10 WS/cm² the beneficial effects of LIPUS are negligible. Alternative minimum efficacious BE values may be, for example, any of at least: 11, 12, 13, 14, 15, 16, 17, 18, 19, 20, 21, 22, 23, 24, or 25 WS/cm²BE or a selected value intermediate between these enumerated values.

In one implementation, the scope of the invention encompasses administration to a testis of a LIPUS application wherein the cells of the testis receive an energetic dosage that is safe, for example, is below a selected maximum safe dosage threshold. In various implementations, the maximum value of the safe threshold value may be a BE value in the range of 40-150 WS/cm². In one embodiment, the maximum value of the safe range is 105 WS/cm²BE. In various embodiments, the maximum safe BE value is selected from the group consisting of 40, 42, 45, 50, 55, 60, 65, 70, 75, 80, 84, 85, 90, 95, 100, 105, 110, 115, 120, 125, 130, 135, 140, 145, or 150 WS/cm² BE, or a selected value intermediate between these enumerated values.

The power density of the applied pulses may be adjusted with the other parameters to arrive at the selected dosage. In various embodiments, the power density is a value between 10 and 500 mW/cm², for example, a value between 25 and 380 mW/cm² In various exemplary embodiments, the power density may be any of 25, 35, 40, 45, 50, 55, 60, 67, 70, 75, 80, 85, 90, 95, 100, 133, 167, 200, 233, 267, 300, or 380 mW/cm², or a selected value intermediate between these enumerated values.

The frequency of pulses may be selected according to the capabilities of the instrument used. For example, in one embodiment, the frequency may be adjusted after selection of the other parameters to arrive at the desired dosage. In various embodiments, the frequency is a value between 500 Hz and 5 MHz, for example, in various embodiments it may be 250, 300, 350, 400, 450, 500, 550, 600, 650, 700, 750, 800, 850, 900, 950, 1000, 1500, 2000, 2500, 3000, 3500 Hz. In one embodiment, a frequency of about 1.0 MHz is used.

The Duty Cycle is a function of the waveform of the delivered ultrasound pulse. Duty Cycle refers to the proportion of the pulse that is “on,” i.e. during which significant energy is emitted. Duty Cycle may be adjusted with the other parameters to arrive at the selected dosage. In one implementation, the Duty Cycle is between 0.2 and 0.5. In various exemplary embodiments, the Duty Cycle value of may be 0.1, 0.2, 0.25, 0.3, 0.35, 0.4, 0.45, 0.5, or greater. In one embodiment, a Duty Cycle value of 0.2 is used.

In a primary implementation, the safe and efficacious dosage values set forth above are applied to a single testis, i.e. one of the two testicles of the scrotum, i.e. the enumerated dosage is that received by one testis. In one embodiment, the application to each testis is divided, with half of the pulses required to attain the selected dosage being applied to one side of one testis and half of the pulses applied to the other side of that testis. The process is repeated with the other testicle, for example, requiring four applications in total to treat both testes. This method minimizes the attenuating effects of intervening and treated tissue, resulting in a more even application to all cells of the target organ. In an alternative implementation, treatment of both testes is administered by two separate applications, one to each testis, for example, by a first application of LIPUS to one side of the scrotum and a second application to the other side of the scrotum. In one embodiment, the safe and efficacious dosages set forth above are applied to the scrotum in a single series of pulses, for example, at double the prescribed dosage of a single testis.

In one implementation, the scope of the invention encompasses a method of treating a low testosterone condition in a subject in need of treatment therefor by the administration of a treatment comprising one or more LIPUS applications to the testes of the subject, wherein the one or more LIPUS applications is administered at a safe and efficacious dosage, wherein the safe and efficacious dosage comprises LIPUS energy received by one testis having a BE value (calculated according to Equation 2) between 10 and 105 WS/cm². In one embodiment, the efficacious and safe dosage comprises LIPUS energy received by each testis has a BE value between 21 and 84 WS/cm². In one embodiment, the efficacious and safe dosage comprises LIPUS energy received by each testis has a BE value between 55 and 65 WS/cm². In one embodiment, the efficacious and safe dosage comprises LIPUS energy received by each testis has a BE value of about 59 WS/cm² BE.

The scope of the invention encompasses various operations of the LIPUS instrument and application of LIPUS to achieve the methods disclosed herein. In one implementation, the scope of the invention encompasses a LIPUS instrument, wherein the LIPUS instrument administers a safe and effective dosage of biologically effective energy to a testis of a subject in the range of 10 to 105 WS/cm²; in one embodiment, the efficacious and safe dosage comprises LIPUS energy having a BE value between 21 and 84 WS/cm²; in one embodiment, the efficacious and safe dosage comprises LIPUS energy having a BE value between 55 and 65 WS/cm²; in one embodiment, the efficacious and safe dosage comprises LIPUS energy having a BE value of about 59 WS/cm².

In one implementation, the scope of the invention encompasses a LIPUS instrument, wherein the LIPUS instrument is configured to administer a safe and effective dosage of biologically effective energy (calculated according to Equation 2) to a testis of a subject in the range of 10 to 105 WS/cm²; in one embodiment, the efficacious and safe dosage comprises LIPUS energy having a BE value between 21 and 84 WS/cm²; in one embodiment, the efficacious and safe dosage comprises LIPUS energy having a BE value between 55 and 65 WS/cm²; in one embodiment, the efficacious and safe dosage comprises LIPUS energy having a BE value of about 59 WS/cm².

In one implementation, the scope of the invention encompasses a method of operating a LIPUS instrument, wherein the LIPUS instrument is operated to administer a safe and effective dosage of biologically effective energy (calculated according to Equation 2) to a testis of a subject in the range of 10 to 105 WS/cm²; in one embodiment, the efficacious and safe dosage comprises LIPUS energy having a BE value between 21 and 84 WS/cm²; in one embodiment, the efficacious and safe dosage comprises LIPUS energy having a BE value between 55 and 65 WS/cm²; in one embodiment, the efficacious and safe dosage comprises LIPUS energy having a BE value of about 59 WS/cm².

In one implementation, the scope of the invention encompasses a LIPUS instrument, for use in a method of treating a low testosterone condition in a subject, the method comprising administering a safe and effective dosage of biologically effective energy (calculated according to Equation 2) to the testes of a subject, wherein the safe and effective dosage of biologically effective energy received by each testis is in the range of 10 to 105 WS/cm²; in one embodiment, the efficacious and safe dosage comprises LIPUS energy having a BE value between 21 and 84 WS/cm²; in one embodiment, the efficacious and safe dosage comprises LIPUS energy having a BE value between 55 and 65 WS/cm²; in one embodiment, the efficacious and safe dosage comprises LIPUS energy having a BE value of about 59 WS/cm².

Treatment Schedules. The treatments of the invention are administered as one or more applications. The applications may be administered according to a selected treatment schedule selected based on the therapeutic needs, subject physiology, convenience of administration, and other parameters.

In one implementation, the subject is administered one application per day at one, two, or three applications per week, with a minimal interval of one day between applications. In one implementation, the subject will receive 1-3 applications per week, with an interval of at least one day between applications. In one embodiment, the application is performed twice per week, with an interval of 2-4 days between applications.

In one implementation, the subject is administered a series of applications, with an interval of weeks between each series. For example, as disclosed herein, the increased testosterone effect of LIPUS treatment may persist for a period of at least six weeks following treatment. In one implementation, a treatment of one or more applications per week is applied, with an interval of one, two, three, four, five, six, seven, eight, nine, or ten weeks between treatments.

In some embodiments, the treatment comprises application of LIPUS is performed at a treatment schedule comprising an application schedule of regular intervals, for example: every other day, every three days, once a week, twice a week, three times a week, etc. wherein the applications are administered over an extended period of weeks, months, or years, or indefinitely.

Two-Sided Treatment. In some implementations, a series of LIPUS applications are applied to multiple sites on the testicular sac in order to achieve a full and relatively even dosage for the majority of the organ. In one embodiment, a portion of the treatment is applied to a first t side of one testicle, then a second portion of the treatment is applied to the other side of that testicle. This is repeated for the opposite testicle.

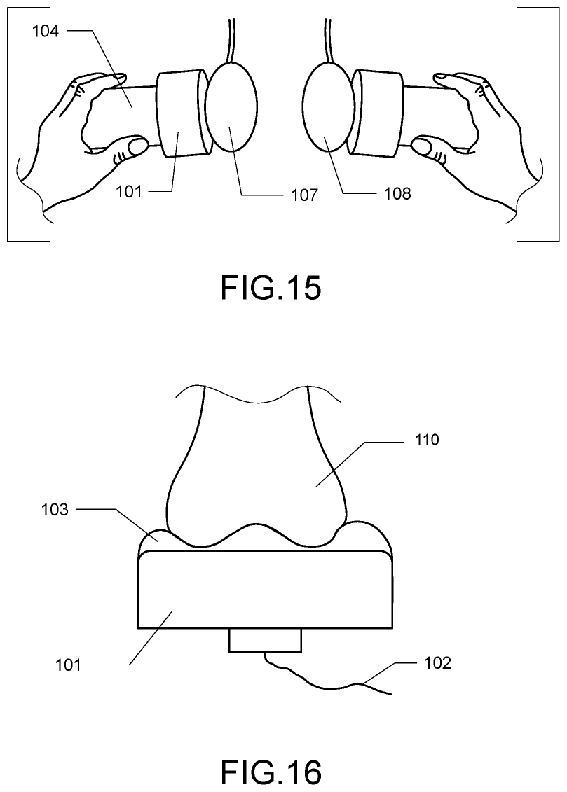

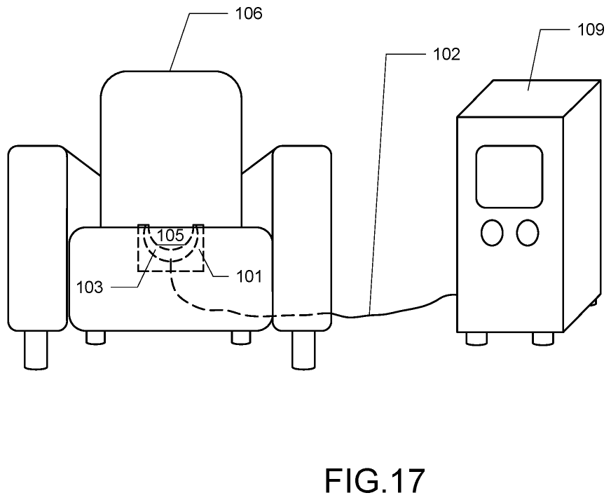

For example, as depicted in FIG. 15 and Table 1, in one implementation, the applied dosage has a BE value of 20. LIPUS is applied in two applications, one to each side of the testes. The attenuation of LIPUS energy in the testes is estimated to be about 38 percent per cm tissue depth. By this scheme, a received dosage of BE value 21 is received.

TABLE 1

| Depth of | BE | BE | |
|---|---|---|---|
| tissue | (WS/cm²) | (WS/cm²) | |
| from left | attenuation | attenuation | Total BE |
| (cm) | from left | from right | (WS/cm²) |
| 0 | 20 | 1 | 21 |
| 0.5 | 16.2 | 4.8 | 21 |
| 1 | 12.4 | 8.6 | 21 |
| 1.5 | 8.6 | 12.4 | 21 |
| 2 | 4.8 | 16.2 | 21 |
| 2.5 | 1 | 20 | 21 |

Methods of Activating Therapeutic Signaling Pathways. The scope of the invention further encompasses methods of activating PERK/ATF4 and Wnt-β-catenin signaling pathways in cells by the application of LIPUS to the cells, for example, by the application of safe and efficacious LIPUS dosages to the target cells, calculated as described herein. In one embodiment, the method encompasses treatment of a selected condition by activation of PERK/ATF4 signaling pathways in target cells by administration of a therapeutically effective dosage of LIPUS to the target cells. In one embodiment, the target cells are cells infected by a virus or other pathogen and administration of LIPUS achieved increased clearance of infected cells, for example, HTLV-1 infected cells. In one embodiment, the target cells are cells of the brain and LIPUS is applied to treat a selected neurodegenerative condition. In one embodiment, the target cells a cancer cell and the LIPUS is applied to promote sensitivity to radiation treatment. In other implementations, the scope of the invention encompasses treatment of a selected condition by activation of Wnt-β-catenin signaling pathways in target cells by administration of a therapeutically effective dosage of LIPUS to the target cells. In various embodiments, the target cells may be hair follicle cells treated with LIPUS to promote hair growth or inhibit hair loss; the target cells may be epdidermal or muscle cells in a wound treated with LIPUS to promote wound healing; or the cells may comprise cells of the brain or central nervous system treated with LIPUS to treat neurodegeneration.

Devices of the Invention. The scope of the invention further encompasses devices configured to deliver LIPUS dosages to the testes. In one embodiment, the device of the invention comprises one or more ultrasonic transducers configured in a receiving body or vessel sized and shaped to envelop the scrotum. In one embodiment, the receiving body comprises a cup, for example, a semicircular or spherical cup, or depression. In one embodiment, the transducer is surrounded by a lip that defines a depression into which the scrotum is placed. In one embodiment, two or more ultrasound transducers are configured in an array, situated in the receiving body to provide LIPUS application evenly across the scrotum.

In a primary embodiment, the device comprises a water bladder, situated between the ultrasonic emitter probe or probe array and the testes. The water bladder (or multiple water bladders side by side) performs dual functions. A first function is to aid in the even application of LIPUS energy to the irregularly shaped and flexible testes. The water bladder will comprise a bag of water, e.g. purified, degassed water, enclosed within a polymeric enclosure, such as rubber, vinyl, or other polymeric material, configured to conform to and envelop the scrotum and to create contact across a substantial portion of the surface of the testicular sac, for the efficient distribution of LIPUS energy across the organ. A second function of the water bladder is to absorb heat to prevent discomfort or injury to the subject receiving the LIPUS treatment.

In one embodiment, the LIPUS instrument comprises a handheld device comprising an emitter that is pressed against a selected target structure of the body.

In one embodiment, the device of the invention is mounted, integrated with, or otherwise present in a chair, stool, or bench that allows the subject to sit or recline while receiving the LIPUS treatments. The scope of the invention may encompass an instrument for the application of a LIPUS treatment to the testes of a subject, comprising one or more ultrasound transducers configured to deliver LIPUS; wherein the one or more ultrasound transducers are mounted in a chair or stool. The ultrasound probe or probe array, optionally covered or enclosed by a water bladder, may be situated in a recessed section of the chair, positioned to align with a sitting subject's scrotum. Alternatively, the emitter of the LIPUS instrument may protrude from the seat of the chair or stool such that it contacts the scrotum.

### EXAMPLES

Example 1. Low-intensity Pulsed Ultrasound Enhances Testosterone Synthesis from rats' testicles. For all experiments, male Sprague-Dawley rats were used in this study. All animals were kept in 12/12 hours light/dark cycle lighting with food and water available.

Experiment 1. Comparison of different treatment methods. To compare the method published by Haddid et al. (referred to in this example as the “Prior Art therapy”), and new methods disclosed herein, two treatments were applied: (1) The Prior Art LIPUS therapy (20 mW/cm², 20 mins every day for 7 days) and (2) The UCSF LIPUS therapy (20 mW/cm², 2 mins twice a week for 3 weeks). For each treatment, two LIPUS frequencies were tested: 1.5 MHz and 1.7 MHz.

Eight male Sprague-Dawley rats at age 36 weeks were divided into five groups: (a). Control group without therapy; (b). Prior art therapy with 1.5 MHz LIPUS (Brz 1.5), 20 mW/cm², 20 mins every day for 7 days; (c) Prior art therapy with 1.7 MHz LIPUS (Brz 1.7), 20 mW/cm², 20 mins every day for 7 days; UCSF therapy with 1.5 MHz LIPUS (UCSF 1.5), 20 mW/cm², 2 mins twice a week for 3 weeks; UCSF therapy with 1.7 MHz LIPUS (UCSF 1.7), 20 mW/cm², 2 mins twice a week for 3 weeks.

Safety study of LIPUS. Fourteen male Sprague-Dawley rats at age 12 weeks were divided into the four groups: control group (control-1, 2 rats), acute safety group (control-2; 1.7 MHz LIPUS L1-L6, 2 min, one-time treatment, 4 rats), chronic effect group LIPUS-1 (1.7 MHz, 67 mW/cm², 4 rats), and LIPUS-2 (1.7 MHz, 86 mW/cm², 4 rats).

Application of LIPUS was performed by using an unfocused LIPUS device. The probe was applied directly to the scrotum of the rat, overlying both testicles, and was coupled to the skin using ultrasound gel (Aquasonic, Parker Laboratories Inc, Fairfield, N.J., USA). Emitted dosage and received dosage were assumed to be equal due to the small size and minimal intervening structure between the emitter and testes of the rat. The LIPUS had a power density of 20 mW/cm² to 333 mW/cm², with a frequency of 1.5 MHz and 1.7 MHz. For the Prior Art therapy group, rats were treated with LIPUS for 20 mins every day for 7 days; for the UCSF therapy group, the rats were treated twice a week for 3 weeks.

One day after the last LIPUS treatment, the rats were sacrificed for serum and organ harvest. Serum testosterone concentrations were measured, and sperm quality was assayed. Histologic examination of the testicular tissue was performed.

Whole blood was aspirated into a collection tube and allowed to clot for 30 minutes. Clotted blood was then centrifuged at 1,000 rpm for 15 minutes. Serum was decanted from the collection tube and stored at −80 centigrade until use. Serum testosterone levels in rats from each group were assayed using the Parameter Testosterone Assay (R&D Systems, Minneapolis, Minn., USA) according to the manufacturer's instructions.

Histological study. Testicular cryosections were prepared, and immunofluorescence was performed as previously described. In brief, tissues were rehydrated with PBS for 5 min and treated with hydrogen peroxide/methanol to quench endogenous peroxidase activity. After rinsing, sections were washed twice in PBS for 5 min followed by 30 min of incubation with 3% horse serum/PBS/0.3% triton X-100. After excess fluid was drained, sections were incubated overnight at 4° C. with primary antibodies including anti-stAR to identify the Leydig cells (StAR; 1:500; Santa Cruz Biotechnologies, Santa Cruz, Calif.; USA), anti-DDX4 (DEAD-Box Helicase 4) to identity germ cells (1:500; Millipore Corp, Bedford, Mass.; USA), and anti-α-smooth muscle (α-SMA) antibody to define the muscle like myeloid cells in the basement membrane of seminiferous tubule (1:1000; Sigma-Aldrich, St. Louis, Mo.; USA). Secondary antibodies used included Alexa-488- and Alexa-594-conjugated antibodies (1:500; Invitrogen). Nuclei were stained with 4′, 6-diamidino-2-phenylindole (DAPI). For image analysis, four randomly selected fields per tissue per animal for each group were photographed and recorded using a Retiga Q Image digital still camera and ACT-1 software (Nikon Instruments Inc., Melville, N.Y., USA).

TUNEL study. After the collected tissue samples were adequately fixed, representative subsections of each sample were then trimmed and placed in tissue cassettes for routine processing. Tissue blocks were prepared from the equatorial area of the testicles. The blocks were transferred to −80° C. before sectioning and then sliced in the thickness of 5 μm. Five slices (sections) were taken for each tissue block and mounted on glass slides, two sections per slide.

Hematoxylin and eosin stain were proceeded for histological evaluation. In the aspects of immunofluorescence, terminal deoxynucleotidyl transferase deoxyuridine triphosphate (dUTP) nick end labeling (TUNEL) assay was performed for the evaluation of apoptosis. Primary antibody with TUNEL assay kit (Abcam, Cambridge, Mass., USA) were incubated in the tissue sections overnight at 4° C. Nuclei were stained with 4′,6-diamidino-2-phenylindole (DAPI) (Invitrogen, Carlsbad, Calif., USA). Apoptosis was measured in terms of the number of apoptotic cells per 1000 cells for the urethra and bladder (apoptotic index or AI).

Microscopic images of tissue sections were also recorded and analyzed. Images were acquired using a bright-light microscope, digital camera and image capture software with Retiga Q image digital camera and ACT-1 software (Nikon Instruments Inc, Melville, N.Y., USA). The quantifications of the images were examined by Image-Pro Plus (Media Cybernetcis, Silver Spring, Md., USA). Measured data (excluding integer scores) was analyzed with one-way ANOVA for statistical comparison. P<0.05 is considered as statistically significant.

RESULTS: Clinical observation and physical examination of experimental animals. No abnormal behavior was observed in any of the rats at any time during the study. No hematuria or bloody stool was seen. No signs of pain were observed from general expression and activities or attention to the treated area. Surveillance showed normal activities with proper response to stimulation of LIPUS treatment, adequate vitality of movement, satisfactory food and water consumption, normal social interaction and usage of the accessories in the cage. No clinical signs of irritation or injury were noted.

Safety study of LIPUS on rat testicles. Grossly, the size of testicles in the Prior therapy groups (Brz 1.5 MHz and 1.7 MHz) is larger than those in the UCSF therapy group (UCSF 1.5 MHz and 1.7 MHz). In histologic sections, the spaces between the seminiferous tubules were very narrow in the Prior therapy group of rat indicative of edema of the testicles and swollen seminiferous tubules.

For the safety studies, 1.7 MHz LIPUS was applied on testicles with different energy levels from L1 to L6 (L1.66 mW/cm²; L2.86 mW/cm²; L3,133 mW/cm²; L4,200 mW/cm²; L5,266 mW/cm²; L6,333 mW/cm²). It was noted that LIPUS at energy level of L5 and L6 caused hemorrhage within the testicle.

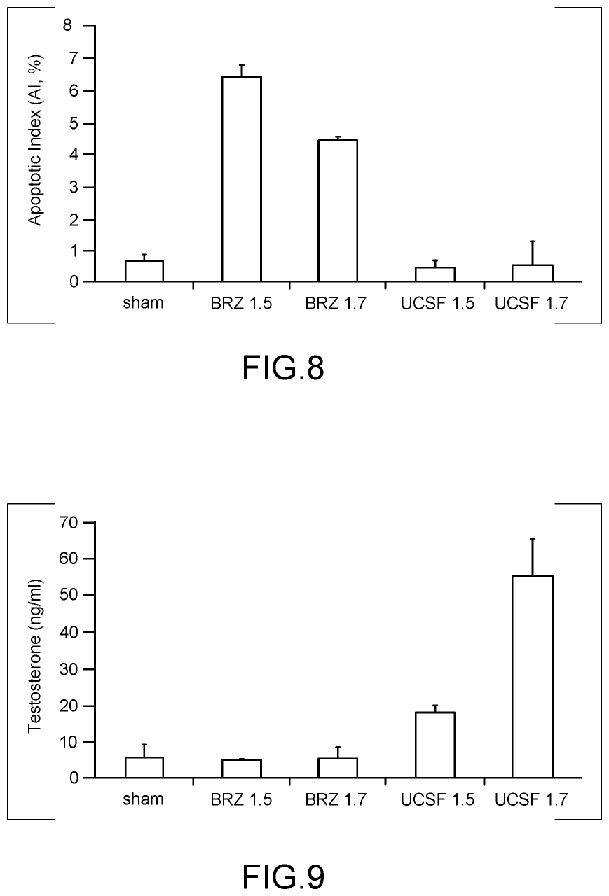

Cellular apoptosis induced by LIPUS. To examine potential harmful effects of LIPUS on testicles, the testicular tissue sections were stained with TUNEL method to identify apoptotic cells. There are more apoptotic cells in the Prior therapy groups, including both Brz 1.5 MHz and Brz 1.7 MHz (FIG. 8.). The apoptotic index (AI) in the Prior therapy groups was much higher than sham control and UCSF therapy groups (6.24+0.36 and 4.44+0.12).

The effects of LIPUS therapy on serum testosterone level. The normal serum testosterone in adult rats is between 2.2±0.2˜7.29±2.02 ng/ml. The result showed that UCSF LIPUS therapy significantly elevated the serum testosterone level to 17.795±6.15 ng/ml at 1.5 MHz and 55.175±10.6 ng/ml at 1.7 MHz respectively. On the other hand, the Prior LIPUS therapy, 20 mins every day for 7 days, did not increase serum testosterone (5.25±0.14 ng/ml and 5.626±2.89 ng/ml at 1.5 MHz and 1.7 MHz respectively). Therefore, the UCSF regenerative LIPUS is good for enhancing serum testosterone (FIG. 9).

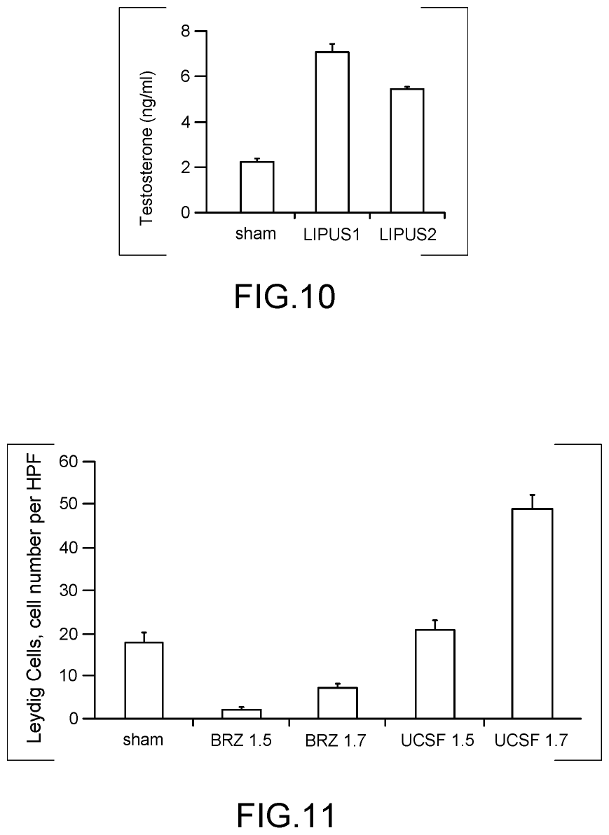

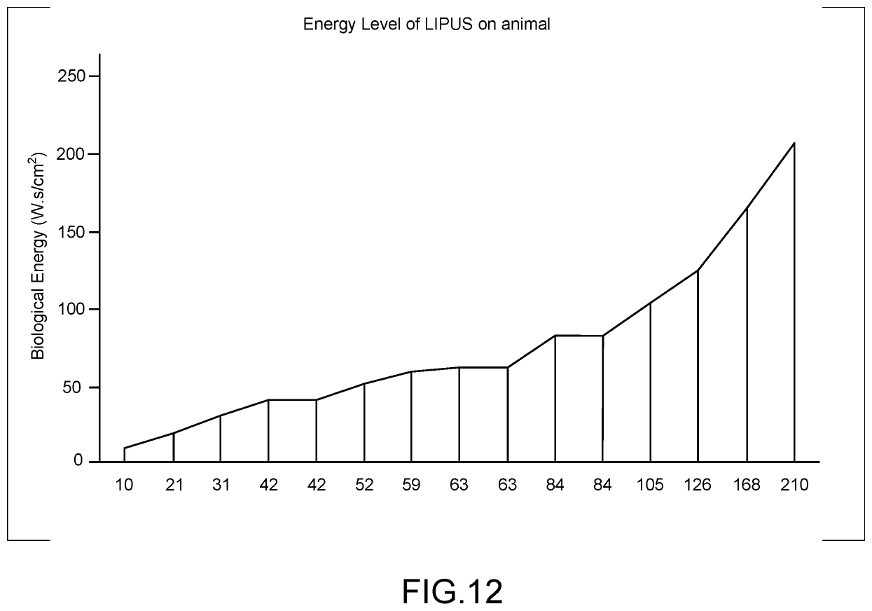

To determine optimal dosages for testosterone production in cultured Leydig cells, it was noted that lower dosage of LIPUS induced higher serum testosterone: dosage of 67 mW/cm² produced testosterone level at 7.1±0.6 ng/ml, while 86 mW/cm² dosage produced 5.4±0.4 ng/ml, wherein both are much higher than the control at 2.2±0.2 ng/ml (FIG. 10).

LIPUS stimulates proliferation of Leydig cells in the testicles. Leydig cells, also known as interstitial cells of Leydig, are found adjacent to the seminiferous tubules in the testicle. They produce testosterone in response to the presence of luteinizing hormone (LH). The UCSF LIPUS therapy significantly increased Leydig cells number in both 1.5 MHz and 1.7 MHz groups but not the Prior LIPUS groups (FIG. 11).

Example 2. Development of BE Dosage and Determination of Safe and Efficacious Dosage Values.

Dosage response of LIPUS on testicle tissues in vivo. The testes of live rats and cultured TM3 and R2C Leydig cells and Sertoli cells (TM4) were treated with a range of LIPUS dosages with varying parameters, as set forth in Table 2.

| | | Power density | T | | | BE |
|---|---|---|---|---|---|---|
| NO | Level | (mW/cm²) | (second) | V | PF | (W · S/cm²) |
| 1 | L1-1 | 66.67 | 60 | 0.2 | 1000 | 10.52232475 |
| 2 | L2-1 | 133.33 | 60 | 0.2 | 1000 | 21.04307123 |
| 3 | L3-1 | 200 | 60 | 0.2 | 1000 | 31.56539597 |
| 4 | L2-2 | 133.33 | 120 | 0.2 | 1000 | 42.08614245 |
| 5 | L4-1 | 266.67 | 60 | 0.2 | 1000 | 42.08772072 |
| 6 | L5-1 | 333.33 | 60 | 0.2 | 1000 | 52.6084672 |
| 7 | L6-1 | 380 | 60 | 0.2 | 1000 | 59.97425235 |
| 8 | L2-3 | 133.33 | 180 | 0.2 | 1000 | 63.12921368 |
| 9 | L3-2 | 200 | 120 | 0.2 | 1000 | 63.13079195 |
| 10 | L2-4 | 133.33 | 240 | 0.2 | 1000 | 84.1722849 |
| 11 | L4-2 | 266.67 | 120 | 0.2 | 1000 | 84.17544144 |
| 12 | L2-5 | 133.33 | 300 | 0.2 | 1000 | 105.2153561 |
| 13 | L2-6 | 133.33 | 360 | 0.2 | 1000 | 126.2584274 |
| 14 | L2-8 | 133.33 | 480 | 0.2 | 1000 | 168.3445698 |
| 15 | L2-10 | 133.33 | 600 | 0.2 | 1000 | 210.4307123 |

Edema was assessed in sections of the treated rat testes with the following scoring system: 0, no edema; +1, rare; +2, moderate; +3, severe edema. In rat testes tissue sections, evident edema was observed at higher doses evidenced by reduced space between the seminiferous tubule.

Apoptosis was assessed in sections of the treated rat testes by the apoptotic cell count on each slide, indicated by positive TUNEL signal under fluorescent microscope in five fields as well as the total cell numbers (DAPI+). The apoptotic index was calculated by tissue apoptosis cells divided by total cells.

Two Leydig cell lines, TM3 and R2C, and one Sertoli cell line (TM4) were cultured under 37° C., 5% CO2 in 1:1 mixture of Ham'S F12 medium and Dulbecco's modified Eagle's medium with 1.2 g/L sodium bicarbonate and 15 mM HEPES; 5% horse serum; 2.5% fetal bovine serum. Cell numbers were assessed by cell counting kit 8 (CCK8) 24 hours following treatment at the varying LIPUS dosages.

Results. In cell culture, application of LIPUS at BE values at 10-42, increased cell proliferation of cultured Leydig cells, while dosages above 42 reduced cell proliferation rate. In cultured Sertoli cells, only BE dosages of 10 WS/cm² stimulated the proliferation of the cells, other BE levels had no observable effect on cell proliferation.

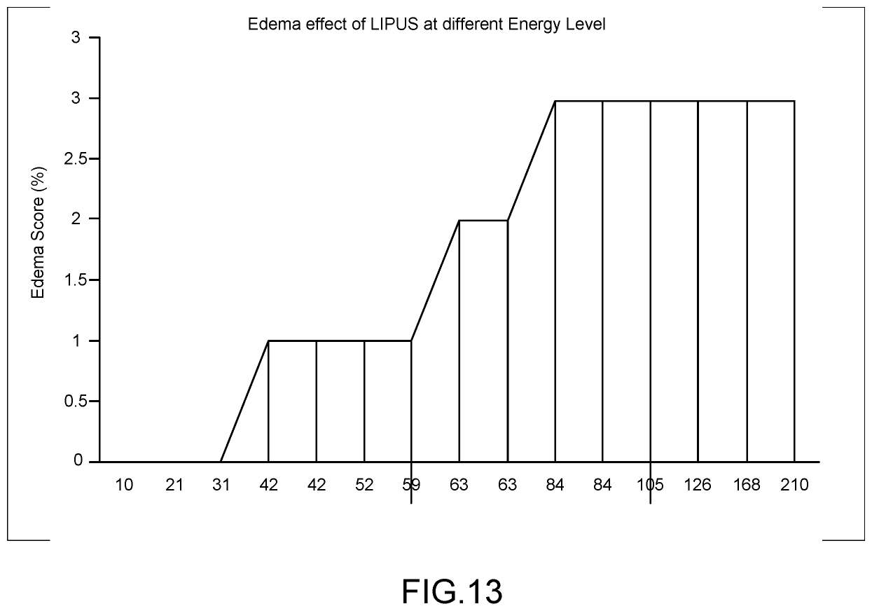

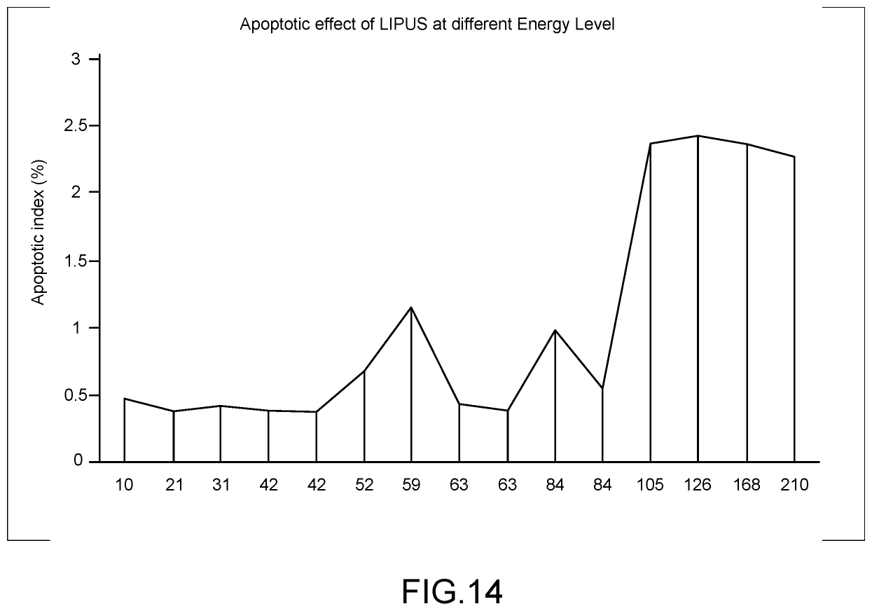

In rat testes, severe edema of testicular tissue was observed at BE dosages of 84 WS/cm² and higher (FIG. 13). In rat testes, abundant apoptosis was observed at BE dosages of 105 WS/cm² and higher (FIG. 14).

Example 3. LIPUS enhancement of testosterone production by cultured Leydig cells. FIG. 24 depicts an overview of the experimental procedure. Mouse TM3 or rat R2C Leydig cells were seeded in 6-well culture plates and grown in FBS medium. The next day, cells were treated with varying LIPUS dosages. 24 hours after treatment, culture media were harvested and cells were analyzed. The number of total cells was measured by DAPI staining, the number of apoptotic cells was measured by TUNEL staining assay, and the testosterone concentration in the culture medium was measured by ELISA androgen assay. LIPUS increased testosterone most significantly at a LIPUS BE dosage value of 10.5 WS/cm2.

Example 4. Overview: Additional experiments were performed to determine the therapeutic energy ranges that stimulate Leydig cell regeneration and increase testosterone production in young SD rats as well as in hypogonadal rats associated with obesity and older age. It was determined that an optimal dosage of LIPUS for rat's testis is 67 mW/cm². When higher energy levels were applied to the testes, tissue edema, hemorrhage and cell death occurred. A stereological study of Leydig cells (conducted as described in van den Driesche, Walker et al. 2012) (Neves, Lorenzini et al. 2017), showed that LIPUS therapy significantly increased Leydig cells number, nuclear volume and cytoplasmic volume. In 10-month-old rats, LIPUS therapy also increased the number of Leydig cells and testicular interstitial blood vessels. In an in vitro pilot experiment, both PERK/ATF4 and Wnt-β-catenin signaling pathways were demonstrated to be active in these processes in TM3 Leydig cells.

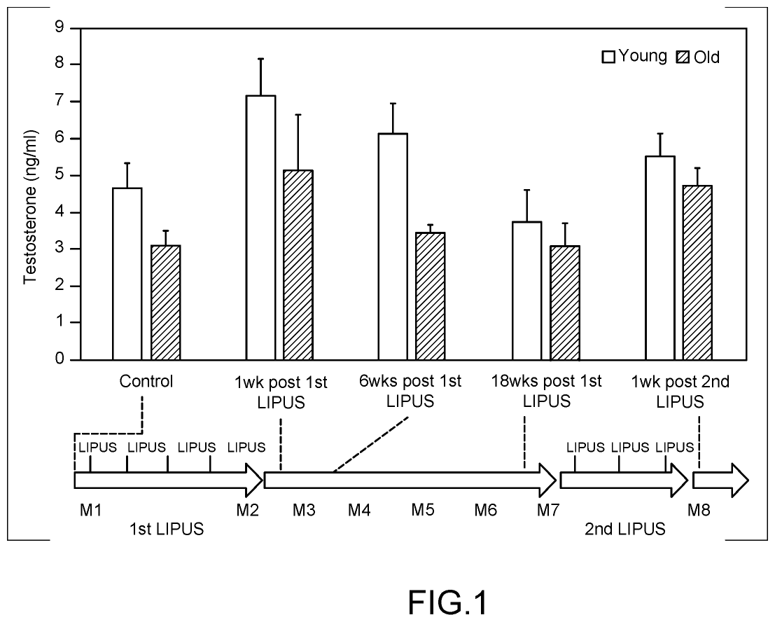

FIG. 1. depicts blood testosterone level (T) over the course of the following experiment, in both young (3 months of age) and old rats (six months of age), treated with LIPUS at a dosage of 67 mW/cm². T was monitored at 1 week, 6 weeks and 18 weeks after the initial LIPUS treatment. The result demonstrated that T was significantly enhanced at 1 week and 6 weeks following the LIPUS in both young and old rats, and declined to normal level at 18 weeks. This result demonstrated that the effect of LIPUS is not permanent. To confirm that this effect is repeatable, a second LIPUS was applied and the result indicated that blood T could be re-enhanced by LIPUS post the first LIPUS treatment.

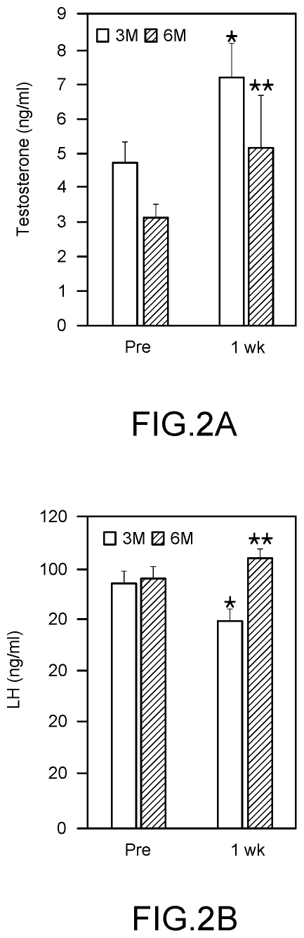

FIGS. 2A and 2B depict serum testosterone and luteinizing hormone (LH) levels following LIPUS therapy. 3-month-old (3M, 3 months of age, n=6) and 6-month-old (6M, six months of age, n=6) male SD rats were used in this experiment. The rats were treated with LIPUS at 67 mW/cm², 2 mins, twice a week for 4 weeks. Serum testosterone (FIG. 2A) and LH (FIG. 2B) levels before (Pre) and 1 week (1 wk) after the last LIPUS therapy were measured. (p<0.05 compared with Pre; **p<0.05 compared with Pre). Increased level of serum testosterone was noted in both 3M and 6M rat groups. However, testosterone negative feedback on LH level was noted in young (3M) but not in older (6M) rats.

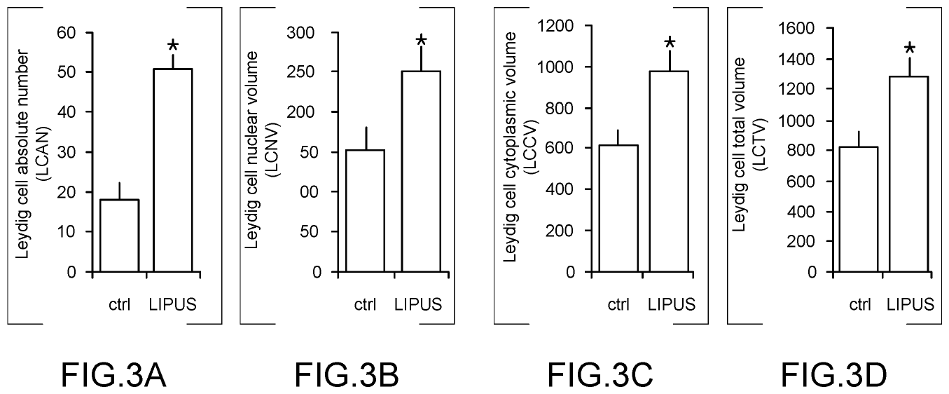

FIG. 3A-3D summarizes the results of a stereological study of Leydig cells following LIPUS therapy. The LIPUS treatment was applied to adult rats (6-month-old) at a dosage of 67 mW/cm², 2 mins, twice a week for 3 weeks. FIG. 3 is a bar chart of Leydig cell nuclear volume (LCNV); FIG. 3B depicts Leydig cell cytoplasmic volume (LCCV); FIG. 3C depicts Leydig cell total volume (LCTV); and FIG. 3D depicts Leydig cell absolute number (LCAN Significant increase in Leydig cell number, nuclear volume, cytoplasmic volume and total cell volume was noted in LIPUS therapy group (n=5, *P<0.05).

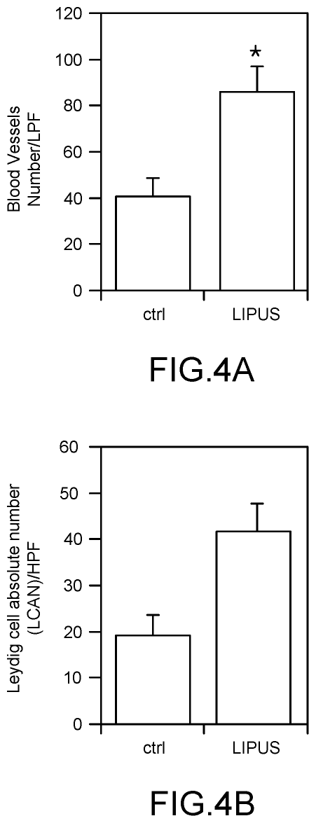

FIGS. 4A and 4B depict testicular blood vessels in older rats treated with LIPUS therapy. The LIPUS therapy was applied to older rats (10-month-old) at a dosage of 67 mW/cm², 2 mins, twice a week for 4 weeks. Tissue sections were stained with 3β-HSD for Leydig cells, Col-IV for blood vessels, and DAPI for cell nuclei. FIG. 4A shows blood vessel number per low-power field (LPF) was significantly higher in the LIPUS therapy group (n=6, *P<0.05). FIG. 4B shows that Leydig cell absolute number (LCAN) per high-power field (HPF) was also significantly increased in the LIPUS therapy group (n=6, *P<0.05).

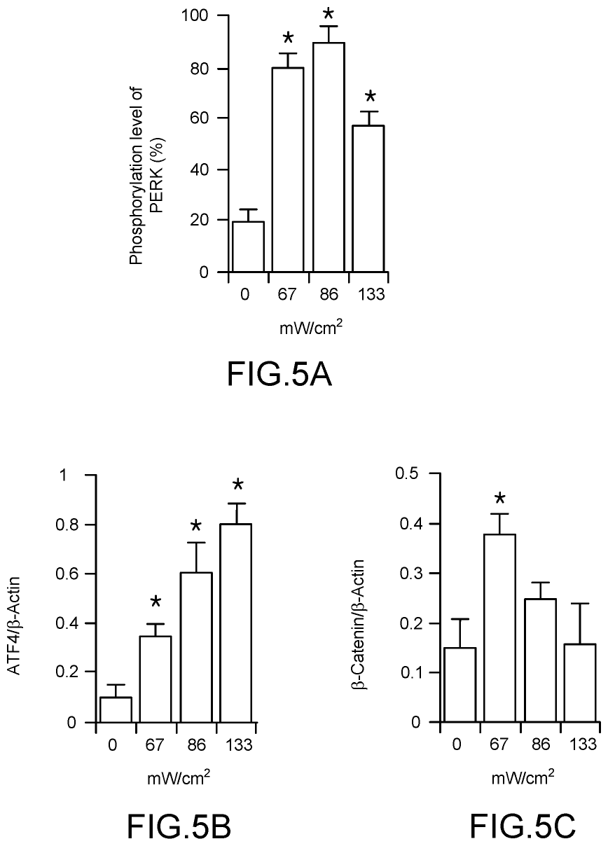

FIGS. 5A, 5B, and 5C. demonstrate that LIPUS activated Wnt and ISR signaling pathway in TM3 Leydig cells in vitro. The TM3 cells were treated with LIPUS at different energy levels at 67, 86, and 133 mW/cm² for 2 mins. 24 hours later the protein was isolated for western blotting. Western blot results of Wnt and ISR related proteins demonstrated effects of LIPUS on signaling pathways. FIG. 5A: phosphorylation level of p-PERK was significantly increased after LIPUS at different energy levels (n=3, *P<0.05). FIG. 5B: IDV (integrated density value) ratio of ATF4 to β-actin was significantly increased after LIPUS at all 3 energy levels (n=3,*P<0.05). FIG. 5C: IDV ratio of β-catenin to β-actin was significantly increased after LIPUS at 67 mW/cm² level only (n=3, *P<0.05). The results suggest that an optimal regenerative therapy level at 67 mW/cm².

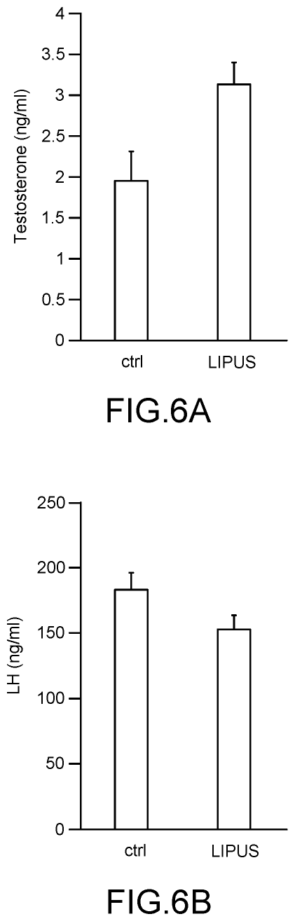

FIGS. 6A and 6B. summarize the effects of LIPUS on serum testosterone and luteinizing hormone (LII) in older male rats. To examine the longer-term effect of LIPUS on testicle, the rats were housed for up to 14 and 16 months of age respectively. The animals were randomly assigned to control and LIPUS-treated groups. LIPUS was applied to both testicles in LIPUS group at 67 mW/cm², 2 mins, twice a week for three weeks. Testosterone (T) and LH were measured 1 week after the last treatment. The results indicated that LIPUS again elevated the serum T levels significantly, while LH was decreased but were still higher than the values at younger age. These results have the following clinical implications: 1) LIPUS-induced elevation of serum testosterone level can be repeated at least 3 times and up to 16 months of age; 2) As compared to young rats, old rats have higher level of luteinizing hormone and lower level of testosterone indicative of decreased Leydig cell sensitivity to luteinizing hormone similar to that of human; 3) In old rats (14-16 months old) LIPUS therapy elevated serum testosterone level and decrease the luteinizing hormone but the levels were still much higher than that of the young rats, indicative of less negative feedback from elevated testosterone level than the young rats and thus suggesting a more pronounced therapeutic effects in older animals. FIG. 6A depicts serum testosterone (a) and FIG. 6B depicts LH levels in control (ctrl, n=6) and LIPUS therapy (LIPUS, n=7) treatments were measured. (p<0.05). Testosterone negative feedback on LH level was noted.

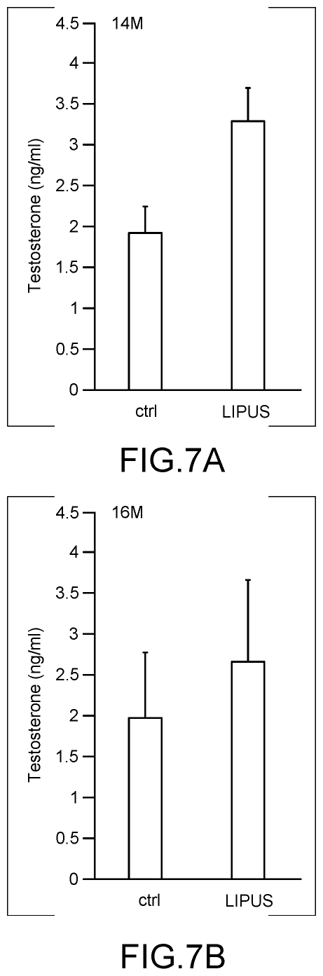

FIG. 7A and FIG. 7B depict the age effect on Serum Testosterone levels following LIPUS therapy. 14-month-old (14M, n=8) and 16-month-old (16M, n=5) male SD rats were used in this experiment. The rats were treated with LIPUS at 67 mW/cm², 2 mins, twice a week for 3 weeks. Serum testosterone in 14M rats (FIG. 7A) and in 16M rats (FIG. 7B) levels 1 week (1 wk) after the last LIPUS therapy were measured. (*p<0.05 in 14M subgroup; **p>0.05 in 16M subgroup).

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of treating a low testosterone condition in a subject in need of treatment therefor by the administration of a treatment comprising one or more LIPUS applications to the testes of the subject, wherein the one or more LIPUS applications is administered at a safe and efficacious dosage;
wherein safe and efficacious received dosage is calculated as
[administered dosage]×[attenuation coefficient], wherein
the administered dosage is calculated as a biologically effective energy value,
wherein the biologically effective energy value=PD×T×PR×PF^{k}
wherein PD is power density per pulse in mW/cm²;
T is the duration of the treatment in seconds;
PR is the pulse ratio of a duty cycle, expressed as a value between 0 and 1;
PF is frequency of pulse delivery, measured in Hz and expressed as a unitless value for purposes of the calculation; and
and k is the coefficient of pulse frequency;
the attenuation coefficient is a value between 0.1 and 1.0;
and the dosage received by each testis is between 10 and 105 WS/cm² biologically effective energy.

2. The method of claim 1, wherein
the low testosterone condition is selected from the group consisting of: age-related reductions in testosterone production; obesity-related reductions in testosterone; primary hypogonadism; congenital hypogonadism; acquired hypogonadism; cryptorchidism; bilateral torsion; orchitis; vanishing testis syndrome; orchiectomy; Klinefelter Syndrome, chemotherapy; toxic damage; hypogonadotropic hypogonadism; gonadotropin or luteinizing hormone-releasing hormone deficiency; pituitary-hypothalamic injury; diabetes; kidney disease; HIV infection; Klinefelter syndrome; Kallman syndrome; and hemochromatosis.

3. The method of claim 1, wherein
the subject has a blood testosterone concentration of less than 300 ng/dl.

4. The method of claim 1, wherein
the subject is an aged male.

5. The method of claim 4, wherein
the male is of an age selected from the group consisting of: over forty years of age; over 45 years of age; over fifty years of age; over fifty five years or age; or over sixty years of age.

6. The method of claim 1, wherein
the subject is an obese subject.

7. The method of claim 1, wherein
the treatment encompasses increasing the muscle mass of the subject.

8. The method of claim 1, wherein
the treatment encompasses improving sexual function of the subject.

9. The method of claim 1, wherein
the treatment encompasses improving fertility of the subject.

10. The method of claim 1, wherein
k is between 0.25 and 0.5

11. The method of claim 10, wherein
k is 0.373.

12. The method of claim 1, wherein
the power density setting of the LIPUS instrument is between 10 and 500 mW/cm².

13. The method of claim 12, wherein
the power density of the LIPUS instrument is between 25 and 380 mW/cm².

14. The method of claim 1, wherein
the frequency of pulse application is between 250 Hz and 5 MHz.

15. The method of claim 14, wherein
the frequency of pulse application is 750 Hz and 1.25 MHz.

16. The method of claim 1, wherein
the duty cycle of the administered waveform is 0.1 to 0.5.

17. The method of claim 16, wherein
the duty cycle is about 0.2.

18. The method of claim 1, wherein
the safe and efficacious dosage is between 21 and 84 WS/cm² biologically effective energy.

19. The method of claim 18, wherein
the safe and efficacious dosage is between 55 and 65 WS/cm² biologically effective energy.

20. The method of claim 19, wherein
the safe and efficacious dosage is about 59 WS/cm² biologically effective energy.

21. The method of claim 1, wherein
the attenuation coefficient is determined according to anatomical attenuation.

22. The method of claim 1, wherein
the attenuation coefficient is determined according to attenuation by structures of the LIPUS delivery device.

23. The method of claim 1, wherein
the attenuation coefficient is a value selected from 0.1, 0.2, 0.3, 0.4, 0.5, 0.6, 0.7, 0.8, 0.9 and 1.0

24. The method of claim 17, wherein
the attenuation coefficient is 1.0.

25. The method of claim 1, wherein
the one or more LIPUS applications are administered no more frequently than every other day.

26. The method of claim 1, wherein
the one or more LIPUS applications are administered one, two, or three times a week.

27. The method of claim 1, wherein
the one or more LIPUS applications are administered in a treatment set comprising one, two or three applications per week with at least one day between applications,
wherein the treatment set is repeated monthly.

28. The method of claim 1, wherein
the one or more applications is applied as a first application to each side of one testis and a second application to the other testis; and
the process is repeated for the other testis.

29. A LIPUS instrument, wherein
the LIPUS instrument administers a safe and effective dosage of biologically effective energy a testis of a subject by the methods of any of claims 1-28.

30. A LIPUS instrument, wherein
the LIPUS instrument administers a safe and effective dosage of biologically effective energy to a testis of a subject in the range of 10 to 105 WS/cm².

31. The method of claim 30, wherein
the efficacious and safe dosage comprises biologically effective energy between 21 and 84 WS/cm².

32. The method of claim 30, wherein
the efficacious and safe dosage comprises biologically effective energy of about 59 WS/cm².

33. A LIPUS instrument, wherein the LIPUS instrument is configured to administer a safe and effective dosage of biologically effective energy to the testes of a subject by the methods of any of claims 1-28.

34. A LIPUS instrument, wherein
the LIPUS instrument is configured to administer a safe and effective dosage of biologically effective energy to a testis of a subject in the range of 10 to 105 WS/cm².

35. The method of claim 34, wherein
the efficacious and safe dosage comprises biologically effective energy between 21 and 84 WS/cm².

36. The method of claim 35, wherein
the efficacious and safe dosage comprises biologically effective energy of about 59 WS/cm².

37. A method of operating a LIPUS instrument, wherein
the LIPUS instrument is operated to administer a safe and effective dosage of biologically effective energy to the testes of a subject by the method of any of claims 1-28.

38. A method of operating a LIPUS instrument, wherein
the LIPUS instrument is operated to administer a safe and effective dosage of biologically effective energy to a testis of a subject in the range of 10 to 105 WS/cm².

39. The method of claim 38, wherein
the efficacious and safe dosage comprises biologically effective energy between 21 and 84 WS/cm².

40. The method of claim 39, wherein
the efficacious and safe dosage comprises biologically effective energy of about 59 WS/cm².

41. A LIPUS instrument, for use in a method of treating a low testosterone condition in a subject by the method of any of claims 1-28.

42. A LIPUS instrument, for use in a method of treating a low testosterone condition in a subject by administering to a testis of the subject a safe and effective dosage of biologically effective energy in the range of 10 to 105 WS/c cm².

43. The LIPUS instrument of claim 42, wherein
the efficacious and safe dosage comprises biologically effective energy between 21 and 84 WS/cm².

44. The LIPUS instrument of claim 43, wherein
the efficacious and safe dosage comprises biologically effective energy of about 59 WS/cm².

45. An instrument for the application of a LIPUS treatment to the testes of a subject, comprising
one or more ultrasound transducers, wherein the one or more transducers are arranged in a receiving body shaped to envelop the scrotum.

46. An instrument for the application of a LIPUS treatment to the testes of a subject, comprising
one or more ultrasound transducers configured to deliver LIPUS; and
a water bladder disposed over the one or more ultrasound transducers.

47. The instrument of claim 46, wherein
the one or more ultrasound transducers and water bladder are arranged in a receiving body shaped to envelop the scrotum.

48. An instrument for the application of a LIPUS treatment to the testes of a subject, comprising
one or more ultrasound transducers configured to deliver LIPUS; wherein
the one or more ultrasound transducers are mounted in a chair or stool.
